(19) United States
(12) Patent Application Publication
(10) Pub. No.: US 2026/0060918 A1
(43) Pub. Date: Mar. 5, 2026

(54) HIGH-PERMEABILITY INCLUSION FOR SOOTHING SKIN REDNESS, HEAT, AND PAIN, AND PREPARATION METHOD AND USE THEREOF

(71) Applicant: N.O.D TOPIA (GUANGZHOU) BIOTECHNOLOGY CO.,LTD., Guangzhou (CN)

(72) Inventors: Jianhua ZHANG, Guangzhou (CN); Shichao LIU, Guangzhou (CN); Wenjiao GUO, Guangzhou (CN)

(73) Assignee: N.O.D TOPIA (GUANGZHOU) BIOTECHNOLOGY CO.,LTD., Guangzhou (CN)

(21) Appl. No.: 19/379,819

(22) Filed: Nov. 5, 2025

Related U.S. Application Data
(63) Continuation of application No. PCT/CN2024/082401, filed on Mar. 19, 2024.

(30) Foreign Application Priority Data
Jun. 9, 2023 (CN) 202310682415.0

(51) Int. Cl. A61K 8/9789; A61K 8/11; A61Q 19/00

(52) U.S. Cl. CPC A61K 8/9789; A61K 8/11; A61Q 19/005; A61K 2800/5922; A61K 2800/596

(57) ABSTRACT

This disclosure relates to a high-permeability inclusion for soothing skin redness, heat, and pain, and a preparation method and use thereof, and belongs to the field of cosmetics. The high-permeability inclusion includes a capsule shell and capsule contents that are encapsulated in a hollow cavity of the capsule shell. The capsule shell includes a nonionic surfactant, a cosurfactant, cholesterol, 4-tert-butylcyclohexanol, a crocus sativus extract, and water. The capsule contents include a Tephrosia purpurea (L.) Pers. seed extract and a Kadsura longipedunculata Finet & Gagnep. extract. In parts by weight, content of each ingredient in the inclusion is as follows: a mass sum of the nonionic surfactant, the cosurfactant, and the cholesterol is 1.6-16.5 parts; a mass sum of the 4-tert-butylcyclohexanol, the crocus sativus extract, the Tephrosia purpurea (L.) Pers. seed extract, and the Kadsura longipedunculata Finet & Gagnep. extract is 0.4-10 parts; and the water is 0.2-4 parts.

### CROSS-REFERENCE TO RELATED APPLICATIONS

The application claims priority to Chinese patent application No. 202310682415.0, filed on Jun. 9, 2023, the entire contents of which are incorporated herein by reference.

### TECHNICAL FIELD

This disclosure belongs to the field of cosmetics, and specifically relates to a high-permeability inclusion for soothing skin redness, heat, and pain, and a preparation method and use thereof.

### BACKGROUND

Sensitive skin (SS) is defined by characteristic perception of skin, including subjective symptoms such as tightness, tingling, burning, and itching. The skin looks normal or is accompanied by injuries such as erythema, scales, and telangiectasia, and inflammatory reactions and skin rashes may occur in severe cases. With the development of industrialization and the improvement of people's cognitive level, a probability of occurrence of sensitive skin is getting higher and higher all over the world. Large-sample surveys in multiple countries show that a prevalence rate of sensitive skin in the world is over 40%, while the prevalence rate of sensitive skin in China is over 38%, with the number of people increasing year by year. Therefore, the sensitive skin has become a social problem to be solved urgently.

A mechanism of sensitive skin is not clear at present, but it is clear that it is a result of many factors. Conventional causes mainly include the following four categories: (1) skin factors: skin barrier destruction, skin nerve imbalance, skin immune imbalance, skin vasodilation, or the like; (2) environmental factors: pollutants, climate, and ultraviolet radiation; (3) lifestyles: cosmetic use, diet, alcohol intake, or the like; and (4) endogenous factors: hormone levels in the body, mental stress, or the like. Currently, types of sensitive skin can be roughly divided into the following three categories: (1) barrier damaged type: the skin appears drying and desquamation, and has high transepidermal water loss (TEWL); (2) immune inflammatory type: the barrier function is normal, but the skin appears inflammation and redness; and (3) neurosensitive type: the barrier function is normal, and there is no inflammation, but the over-expression of transient receptor potential vanilloid 1 (TRPV1), which is a heat and pain sensory channel of the skin, causes nerve tingling, burning, and redness. However, no matter how it is classified, sensitive skin is closely related to barriers, inflammation, overexpression of nerve, and external environmental pressure stimulation. These causes often promote and affect each other, and produce persistent redness, heat, and pain, making sensitive skin difficult to cure.

Currently, commercial soothing products have the following shortcomings: (1) it only aims at a single symptom of redness, heat, or pain, and is impossible to address them simultaneously and effectively; (2) discomforts such as redness, heat, and pain caused by sensitive skin are a major source of troubles for consumers, and most products cannot suppress the discomforts immediately, resulting in poor experience; (3) the more active the raw materials are, the more likely they are to produce discoloration, flavor reversion, or inactivation, and it is difficult for them to achieve good compatibility after being added to a formula; and (4) sensitive skin involves not only the epidermal layer, but also the corium layer, due to the existence of skin barrier, it is difficult for active ingredients to penetrate into effect targets of the deep skin, resulting in poor effect.

### SUMMARY

An objective of this disclosure is to overcome the problems existing in the above-mentioned prior art, and provide a high-permeability inclusion for soothing skin redness, heat, and pain and a preparation method and use thereof, which can achieve the effects of soothing sensitive skin instantly and relieving redness, heat, and pain for a long time.

This disclosure is achieved by the following technical solutions:
This disclosure provides a high-permeability inclusion for soothing skin redness, heat, and pain. The inclusion includes a capsule shell and capsule contents that are encapsulated in a hollow cavity of the capsule shell;
the capsule shell includes a nonionic surfactant, a cosurfactant, cholesterol, 4-tert-butylcyclohexanol, a crocus sativus extract, and water;
the capsule contents include a Tephrosia purpurea (L.) Pers. seed extract and a Kadsura longipedunculata Finet & Gagnep. extract;
in parts by weight, content of each ingredient in the inclusion is as follows:
a mass sum of the nonionic surfactant, the cosurfactant, and the cholesterol is 1.6-16.5 parts;
a mass sum of the 4-tert-butylcyclohexanol, the crocus sativus extract, the Tephrosia purpurea (L.) Pers. seed extract, and the Kadsura longipedunculata Finet & Gagnep. extract is 0.4-10 parts; and the water is 0.2-4 parts.

The high-permeability inclusion of this disclosure is compounded with various active ingredients to form a vesicle structure, so that the inclusion has good skin applicability and permeability, can achieve the effects of soothing sensitive skin instantly and relieving redness, heat, and pain for a long time, and has good anti-inflammatory and skin barrier repairing effects. In the inclusion of this disclosure, the 4-tert-butylcyclohexanol is an inhibitor of the transient receptor potential vanilloid 1 (TRPV1), which can reduce calcium ion flux through the TRPV1, quickly soothe the nerve sensory overreaction of sensitive skin, obviously reduce burning and tingling sensation, and reduce sensitive symptoms. A main component of the crocus sativus extract is glycosylated flavonoid, which is an agonist of the cannabinoid type II receptor (CB2). The activation of the CB2 can weaken the activation of the TRPV1, inhibit inflammatory reactions, promote the release of “β-endorphin”, have analgesic effect and obvious barrier repair effect, and can improve uncomfortable symptoms of sensitive skin. The Tephrosia purpurea (L.) Pers. seed extract can promote the production of a “neurosoothing peptide”, that is the β-endorphin, activate the expression of anti-stress genes in skin cells, and has a significant inhibitory effect on skin irritation caused by external pressure. The Kadsura longipedunculata Finet & Gagnep. extract has dual effects of soothing irritation and evening out skin color, can reduce neuroinflammation and hemangiectasis, reduce skin redness, and soothe the sensitive skin by regulating nonspecific vascular and neuroinflammation, and can limit the diffusion of pigmentation caused by ultraviolet rays, thus making the skin color even. Through combination of the 4-tert-butylcyclohexanol, the crocus sativus extract, the Tephrosia purpurea (L.) Pers. seed extract, and the Kadsura longipedunculata Finet & Gagnep. extract as active substances, the 4-tert-butylcyclohexanol and the crocus sativus extract in the capsule shell structure are preferentially released, which can realize instant and long-lasting soothing, inhibit the release of inflammatory factors, soothe skin irritation in an all-round way, improve skin redness and inflammation, and increase skin tolerance.

As a preferred embodiment of the high-permeability inclusion for soothing skin redness, heat, and pain, a mass ratio of the 4-tert-butylcyclohexanol, the crocus sativus extract, the Tephrosia purpurea (L.) Pers. seed extract, and the Kadsura longipedunculata Finet & Gagnep. extract is (0.1-3):(0.1-2):(0.1-2):(0.1-3). This disclosure has found through research that four active ingredients have better effects of soothing redness, heat, and pain under the above ratio.

Preferably, the mass ratio of the 4-tert-butylcyclohexanol, the crocus sativus extract, the Tephrosia purpurea (L.) Pers. seed extract, and the Kadsura longipedunculata Finet & Gagnep. extract is (0.5-1):(0.5-1):(0.1-1):(0.5-1).

More preferably, the mass ratio of the 4-tert-butylcyclohexanol, the crocus sativus extract, the Tephrosia purpurea (L.) Pers. seed extract, and the Kadsura longipedunculata Finet & Gagnep. extract is 1:0.5:0.75:0.5. When these four active ingredients are mixed in this specific ratio, the effect of soothing skin redness, heat, and pain is better and the benefits are higher.

As a preferred embodiment of the high-permeability inclusion for soothing skin redness, heat, and pain, a mass ratio of the nonionic surfactant, the cosurfactant, and the cholesterol is (1-12):(0.1-1.5):(0.5-3). At the above ratio, the obtained vesicle inclusion can better embed active ingredients, which makes the obtained high-permeability inclusion more stable.

More preferably, the mass ratio of the nonionic surfactant, the cosurfactant, and the cholesterol is 6:0.75:1.5. At the specific ratio, the obtained vesicle inclusion can have a better embedding effect, and the obtained high-permeability inclusion has better stability.

As a preferred embodiment of the high-permeability inclusion for soothing skin redness, heat, and pain, at least one of the following is met:
(1-1) the nonionic surfactant includes at least one of monoglyceride, propylene glycol fatty acid esters, polyglyceryl-10 oleate, polyglyceryl-10 diisostearate, polyglyceryl-3 methylglucose distearate, polyglyceryl-10 laurate, glyceryl oleate citrate, polyglyceryl-2 dipolyhydroxystearate, cetearyl alcohol, and cetearyl glucoside; and
(1-2) the cosurfactant includes at least one of polyethylene glycol-400, polyethylene glycol-600, caprylic/capric triglyceride, polyethylene glycol-40 (PEG-40) hydrogenated castor oil, and polyethylene glycol-60 (PEG-60) hydrogenated castor oil.

Preferably, at least one of the following is met:
(2-1) the nonionic surfactant is the monoglyceride, the cetearyl alcohol, or the polyglyceryl-10 oleate; and
(2-2) the cosurfactant is the PEG-40 hydrogenated castor oil.

Further, in the nonionic surfactant, a mass ratio of the monoglyceride, the polyglyceryl-10 oleate, and the cetearyl alcohol is 3:2:1.

Another objective of this disclosure is to provide a preparation method of the high-permeability inclusion for soothing skin redness, heat, and pain, including the following steps:
S1: stirring the 4-tert-butylcyclohexanol, the crocus sativus extract, the nonionic surfactant, and the cosurfactant under a heating condition until they were completely mixed, and cooling them to obtain a nonionic surfactant mixture;
S2: mixing the nonionic surfactant mixture obtained in step S1 with water and cholesterol, and performing heating, homogenizing, and cooling on them to obtain capsule materials; and
S3: adding the Tephrosia purpurea (L.) Pers. seed extract and the Kadsura longipedunculata Finet & Gagnep. extract into the capsule materials obtained in step S2, and homogenizing them under high pressure to obtain the high-permeability inclusion for soothing skin redness, heat, and pain.

In this disclosure, the preparation method of the high-permeability inclusion is simple and feasible, has high stability, and is suitable for large-scale industrial production. In addition, in this disclosure, the nonionic surfactant is further used to encapsulate the 4-tert-butylcyclohexanol and the crocus sativus extract in the capsule shell layer and encapsulate the Tephrosia purpurea (L.) Pers. seed extract and the Kadsura longipedunculata Finet & Gagnep. extract in an aqueous cavity in the capsule; the 4-tert-butylcyclohexanol and the crocus sativus extract are preferentially released after contacting the skin, and act on the transient receptor potential vanilloid 1 (TRPV1) and the cannabinoid type II receptor (CB2), which can immediately inhibit the transmission of related red, heat, and painful sensations; and then the Tephrosia purpurea (L.) Pers. seed extract and the Kadsura longipedunculata Finet & Gagnep. extract are under targeting delivery to action sites, so as to continuously soothe the discomforts of the skin and achieve better instant and long-lasting soothing effects.

As a preferred embodiment of the preparation method of the high-permeability inclusion for soothing skin redness, heat, and pain, at least one of the following is met:
(3-1) in step S1, a heating temperature is 60° C. to 70° C.;
(3-2) in step S2, a heating temperature is 70° C. to 80° C., and a rotating speed during homogenization is 1500 r/min to 2500 r/min; and
(3-3) in step S3, a pressure of the high-pressure homogenization is 500 bar to 800 bar, a rotating speed is 1000 r/min to 2000 r/min, and a number of rotating times is 3 to 5.

Still another objective of this disclosure is to provide use of the high-permeability inclusion for soothing skin redness, heat, and pain in cosmetics.

The high-permeability inclusion of this disclosure has comprehensive instant and long-lasting effects of soothing skin redness, heat, and pain. Due to the formation of a vesicle encapsulation structure, the inclusion can reach a micron level in size, has good permeability, is beneficial to skin absorption, and can be widely used in cosmetics, including but not limited to cosmetic water, emulsion, facial cream, facial mask, essence, spray, sunscreen cream, sunscreen spray, sunscreen lotion, and other skin care products and various make-up products.

Yet another objective of this disclosure is to provide an emulsion. The emulsion includes the high-permeability inclusion for soothing skin redness, heat, and pain according to this disclosure, and a mass percentage of the inclusion for soothing skin redness, heat, and pain in the emulsion is 3.2%-27%.

The high-permeability inclusion of this disclosure is applied to the emulsion, a soothing emulsion with instant and long-lasting soothing effects on skin redness, heat, and pain can be obtained.

Preferably, the emulsion includes the following components in percentage by mass: 3.2%-27% of the high-permeability inclusion for soothing skin redness, heat, and pain in this disclosure, 0.1%-0.5% of thickener, 4%-20% of moisturizer, 0.1%-10% of emulsifier, 0.5%-5% of preservative, 0.05%-0.2% of essence, 0.05-0.1% of pH regulator, and water as the remainder.

Compared with the prior art, this disclosure has the following beneficial effects:
(1) in this disclosure, the 4-tert-butylcyclohexanol, the crocus sativus extract, the Tephrosia purpurea (L.) Pers. seed extract, and the Kadsura longipedunculata Finet & Gagnep. extract are scientifically compounded to form a high-permeability inclusion for comprehensively soothing skin redness, heat, and pain. As an antagonist of the TRPV1, the 4-tert-butylcyclohexanol can reduce the heat and pain caused by external environment stimulation; as an activator of the CB2, the crocus sativus extract can continuously ease pain; the Tephrosia purpurea (L.) Pers. seed extract can generate the “natural analgesic”, that is β-endorphin, for the discomforts caused by external pressure; and the Kadsura longipedunculata Finet & Gagnep. extract can inhibit vascular inflammation and hemangiectasis, and correct couperose skin. Moreover, the high-permeability inclusion also has good anti-inflammatory and skin barrier repairing effects, and can soothe irritation in an all-round way and improve skin tolerance.
(2) in the preparation method of this disclosure, the nonionic surfactant is further used to encapsulate the 4-tert-butylcyclohexanol and the crocus sativus extract in the capsule shell layer and encapsulate the Tephrosia purpurea (L.) Pers. seed extract and the Kadsura longipedunculata Finet & Gagnep. extract in an aqueous cavity in the capsule; the 4-tert-butylcyclohexanol and the crocus sativus extract are preferentially released after contacting the skin, and act on the transient receptor potential vanilloid 1 (TRPV1) and the cannabinoid type II receptor (CB2), which can immediately inhibit the transmission of related red, heat and painful sensations, and achieve instant soothing and fading of redness; and then the Tephrosia purpurea (L.) Pers. seed extract and the Kadsura longipedunculata Finet & Gagnep. extract are under targeting delivery to action sites, so as to continuously soothe the discomforts of the skin, and compared with liposomes prepared from cholesterol and phospholipids, the obtained vesicle inclusion is more stable.
(3) the high-permeability inclusion of this disclosure can encapsulate a plurality of soothing active ingredients together to jointly generate beneficial effects. When used as a drug reservoir, the vesicle inclusion has a certain viscosity, which can prolong the retention time of the drug in the skin. The nonionic surfactant in the vesicle inclusion can relax cells in the stratum corneum and enhance the penetration ability of the drug into the epidermis and dermis. Compared with the use of penetration enhancers, the vesicle inclusion does not cause irritation or other harm to tissues, is low in cost, and is suitable for industrial production.

### DETAILED DESCRIPTION OF THE EMBODIMENTS

In order to better illustrate the objectives, technical solutions, and advantages of this disclosure, this disclosure is described in further detail below with reference to the specific embodiments. A person skilled in the art should be understood that the specific embodiments described herein are merely used to explain this disclosure but not to limit this disclosure.

Experimental methods in the embodiments, unless otherwise specified, are all conventional methods; and materials and reagents used, unless otherwise specified, are all commercially available. Crocus sativus extracts come from a French ID BIG company, Tephrosia purpurea (L.) Pers. seed extracts come from a Givaudan company in Switzerland, and Kadsura longipedunculata Finet & Gagnep. extracts come from Shanghai Benco Industrial Co., Ltd.

Inclusions for soothing skin redness, heat, and pain in Examples 1 to 8 and inclusions in Comparative Examples 1 to 9 are made of components in parts by weight in Table 1.

TABLE 1
Components in parts by weight (parts)

| | | | | | | | | | | Compar- |
|---|---|---|---|---|---|---|---|---|---|---|
| | | | | | | | | | | ative |
| | | Exam- | Exam- | Exam- | Exam- | Exam- | Exam- | Exam- | Exam- | Exam- |
| Component | | ple 1 | ple 2 | ple 3 | ple 4 | ple 5 | ple 6 | ple 7 | ple 8 | ple 1 |
| Active | 4-tert- | 1 | 0.5 | 1 | 0.1 | 3 | 1 | 0.5 | 1 | 1 |
| substance | butylcyclohexanol | | | | | | | | | |
| | Crocus sativus | 0.5 | 0.5 | 1 | 0.1 | 2 | 0.5 | 0.5 | 0.5 | 0.5 |
| | extract | | | | | | | | | |
| | Tephrosia | 0.75 | 0.1 | 1 | 0.1 | 2 | 0.75 | 0.5 | 0.75 | 0.75 |
| | purpurea (L.) | | | | | | | | | |
| | Pers. seed | | | | | | | | | |
| | extract | | | | | | | | | |
| | Kadsura | 0.5 | 0.5 | 1 | 0.1 | 3 | 0.5 | 0.5 | 0.5 | 0.5 |
| | longipedunculata | | | | | | | | | |
| | Finet & Gagnep. | | | | | | | | | |
| | extract | | | | | | | | | |
| Nonionic | Monoglyceride | 3 | 3 | 3 | 0.5 | 6 | 4.5 | 3 | 3 | 3 |
| surfactant | Polyglyceryl- | 2 | 2 | 2 | 0.34 | 4 | 3.5 | 2 | 2 | 2 |
| | 10 oleate | | | | | | | | | |
| | Cetearyl | 1 | 1 | 1 | 0.16 | 2 | 1.5 | 1 | 1 | 1 |
| | alcohol | | | | | | | | | |
| Cosurfactant | PEG-40 | 0.75 | 0.75 | 0.75 | 0.1 | 1.5 | 0.75 | 0.75 | 1 | 2 |
| | hydrogenated | | | | | | | | | |
| | castor oil | | | | | | | | | |

| | | | | | | | | | |
|---|---|---|---|---|---|---|---|---|---|
| Cholesterol | 1.5 | 1.5 | 1.5 | 0.5 | 3 | 1.5 | 1.5 | 1 | 4 |
| Deionized | 2 | 2 | 2 | 2 | 2 | 2 | 2 | 2 | 2 |
| water | | | | | | | | | |
| Lecithin | / | / | / | / | / | / | / | / | / |

| | | Compar- | Compar- | Compar- | Compar- | Compar- | Compar- |
|---|---|---|---|---|---|---|---|
| | | ative | ative | ative | ative | ative | ative |
| | | Exam- | Exam- | Exam- | Exam- | Exam- | Exam- |
| Component | | ple 2 | ple 3 | ple 6 | ple 7 | ple 8 | ple 9 |
| Active | 4-tert- | 4 | 1 | 1 | 0.1 | 1.5 | / |
| substance | butylcyclohexanol | | | | | | |
| | Crocus sativus | 2.5 | 0.5 | 0.5 | 0.05 | / | 1.5 |
| | extract | | | | | | |
| | Tephrosia | 2.5 | 0.75 | 0.75 | 0.075 | 0.75 | 0.75 |
| | purpurea (L.) | | | | | | |
| | Pers. seed | | | | | | |
| | extract | | | | | | |
| | Kadsura | 3 | 0.5 | 0.5 | 0.05 | 0.5 | 0.5 |
| | longipedunculata | | | | | | |
| | Finet & Gagnep. | | | | | | |
| | extract | | | | | | |
| Nonionic | Monoglyceride | 3 | / | 3 | 3 | 3 | 3 |
| surfactant | Polyglyceryl- | 2 | / | 2 | 2 | 2 | 2 |
| | 10 oleate | | | | | | |
| | Cetearyl | 1 | / | 1 | 1 | 1 | 1 |
| | alcohol | | | | | | |
| Cosurfactant | PEG-40 | 0.75 | 0.75 | 0.1 | 0.75 | 0.75 | 0.75 |
| | hydrogenated | | | | | | |
| | castor oil | | | | | | |

| | | | | | | |
|---|---|---|---|---|---|---|
| Cholesterol | 1.5 | 1.5 | 0.3 | 1.5 | 1.5 | 1.5 |
| Deionized | 2 | 2 | 2 | 2 | 2 | 2 |
| water | | | | | | |
| Lecithin | / | 6 | / | / | / | / |

The preparation method of the high-permeability inclusion for soothing skin redness, heat, and pain in Examples 1 to 8 and in Comparative Examples 1 to 2 and Comparative Examples 6 to 7 was as follows:
S1: mixing the 4-tert-butylcyclohexanol, the crocus sativus extract, the monoglyceride, the polyglyceryl-10 oleate, the cetearyl alcohol, and PEG-40 hydrogenated castor oil, heating them to 70° C., stirring them at a low speed until they were completely mixed, and cooling them to room temperature to obtain a nonionic surfactant mixture;
S2: heating the nonionic surfactant mixture obtained in step S1 with deionized water and cholesterol to 80° C., homogenizing for 10 min at a rotating speed of 2,500 r/min, and cooling them to room temperature to obtain capsule materials; and
S3: adding the Tephrosia purpurea (L.) Pers. seed extract and the Kadsura longipedunculata Finet & Gagnep. extract into the capsule materials obtained in step S2, homogenizing with micro-jet at high pressure, homogenizing at a speed of 2,000 r/min and a pressure of 800 bar for five times, and obtaining the high-permeability inclusion for soothing skin redness, heat, and pain.

### Example 9

Example 9 differs from Example 1 only in that the nonionic surfactant was 3 parts of polyglyceryl-10 diisostearate, 2 parts of glyceryl oleate citrate, and 1 part of polyglyceryl-10 laurate, the cosurfactant was 0.75 part of caprylic/capric triglyceride, and the remaining components and ratio were the same as those in Example 1. In the preparation method, only the nonionic surfactant and the cosurfactant were replaced by the components of this example, and the remaining were the same as those in Example 1, so as to obtain the inclusion in this example.

### Comparative Example 3

The preparation method of the inclusion for soothing skin redness, heat, and pain in Comparative Example 3 was as follows:
S1: adding the 4-tert-butylcyclohexanol, the crocus sativus extract, the lecithin, the cholesterol, and an appropriate amount of anhydrous ethanol into a round-bottomed flask, fully diffusing them under ultrasonic conditions, evaporating them in a rotary evaporator at a temperature of 30° C.-50° C., and then drying them in a vacuum drying oven to form film-like capsule materials; and
S2: adding the Tephrosia purpurea (L.) Pers. seed extract, the Kadsura longipedunculata Finet & Gagnep. extract, and PEG-40 hydrogenated castor oil into the round-bottomed flask for hydration, continuing water bath ultrasonic diffusion, homogenizing the mixed solution with micro-jet at high pressure, homogenizing at a speed of 2000 r/min and a pressure of 800 bar, and removing impurities to obtain the inclusion of Comparative Example 3.

### Comparative Example 4

The inclusion in Comparative Example 4 had same component and content as the high-permeability inclusion in Example 1, but the preparation method was different. In this Comparative Example, only all the components in the inclusion were stirred and mixed evenly to obtain the inclusion in Comparative Example 4.

### Comparative Example 5

Comparative Example 5 differs from Example 1 only in that the preparation method of the inclusion was different. The preparation method of the inclusion in Comparative Example 5 for soothing skin redness, heat, and pain was as follows:
S1: mixing the 4-tert-butylcyclohexanol, the monoglyceride, the polyglyceryl-10 oleate, the cetearyl alcohol, and PEG-40 hydrogenated castor oil, heating them to 70° C., stirring them at a low speed until they were completely mixed, and cooling them to room temperature to obtain a nonionic surfactant mixture;
S2: heating the nonionic surfactant mixture obtained in step S1 with deionized water and cholesterol to 80° C., homogenizing for 10 min at a rotating speed of 2,500 r/min, and cooling them to room temperature to obtain capsule materials; and
S3: adding the crocus sativus extract, the Tephrosia purpurea (L.) Pers. seed extract, and the Kadsura longipedunculata Finet & Gagnep. extract into the capsule materials obtained in step S2, homogenizing with micro-jet at high pressure, homogenizing at a speed of 2,000 r/min and a pressure of 800 bar for five times, and obtaining the inclusion for soothing skin redness, heat, and pain.

In the preparation method of Comparative Example 8, preparation parameters were the same as those in Example 1, except that the crocus sativus extract was not added.

In the preparation method of Comparative Example 9, preparation parameters were the same as those in Example 1, except that the 4-tert-butylcyclohexanol was not added.

The inclusions in Examples and Comparative Examples were added into an emulsion to prepare an emulsion product, which was made of the following components in percentage by weight in Table 2:

TABLE 2
Mass percentage of each component in emulsion

| Phase | Component | Name of raw material | Ratio |
|---|---|---|---|
| A | Moisturizer | Glycerol | 4% |
| | | Trehalose | 0.5% |
| | | Low molecular weight sodium | 0.05% |
| | | hyaluronate | |
| | Thickener | Carbomer 980 | 0.1% |
| B | Emulsifier | C14-22 alcohol/C12-20 alkyl | 4.5% |
| | | glucoside | |
| | | Caprylic/capric triglyceride | 2.5% |
| | | Polydimethylsiloxane | 2% |
| C | Preservative | 1,3-propanediol | 2% |
| | | 1,2-hexanediol | 2% |
| | | p-hydroxyacetophenone | 0.2% |
| D | Inclusion | Inclusions in Examples and | 19% |
| | | Comparative Examples | |
| | Essence | Essence | 0.1% |
| | pH regulator | Arginine | 0.15% |
| | Water | Deionized water | Balance |

The preparation method of the emulsion was as follows: putting the components in the phase A into water in turn, fully swelling the carbomer 980, then homogenizing and mixing evenly, respectively heating with the phase B, mixing, homogenizing, and emulsifying at 80° C., heating the phase C until the p-hydroxyacetophenone was melted, adding into the emulsion and evenly stirring, after the temperature was reduced to 40° C., adding the phase D and stirring to form a uniform emulsion, and finally adding the arginine to adjust the pH to obtain the emulsion.

### Effect Example 1: Stability Experiment

The emulsions obtained in Examples 1 to 9 and Comparative Examples 1 to 9 were tested for stability, and test results were shown in Table 3.

Test method: samples were put in a constant temperature device at 50° C., 40° C., 25° C., 5° C., and −8° C. and a UV light aging box for stability testing, and the samples were taken out on the 0D, 1D, 7D, 14D, 21D, and 30D respectively to observe whether there were obvious abnormal appearance changes such as discoloration, delamination, sedimentation, and precipitation.

TABLE 3
Stability test results

| | Test | | | | | | | |
|---|---|---|---|---|---|---|---|---|
| Examples | conditions | | D 0 | D 3 | D 7 | D 14 | D 21 | D 30 |
| Example 1 | UV | | Normal | | | | | |
| | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 2 | UV | | Normal | | | | | |
| | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 3 | UV | | Normal | | | | | |
| | 50° | C. | Normal | Normal | Normal | Normal | Slight | Yellowing |
| | | | | | | | yellowing | deepened |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 4 | UV | | Normal | | | | | |
| | 50° | C. | Normal | Normal | Normal | Normal | Normal | Normal |
| | 40° | C. | Normal | Normal | Normal | Normal | Normal | Normal |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 5 | UV | | Normal | | | | | |
| | 50° | C. | Normal | Normal | Normal | Normal | Slight | Yellowing |
| | | | | | | | yellowing | deepened |
| | 40° | C. | Normal | Normal | Normal | Normal | Normal | Normal |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 6 | UV | | Slight yellowing | | | | | |
| | 50° | C. | Normal | Normal | Normal | Normal | Slight | Yellowing |
| | | | | | | | yellowing | deepened |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 7 | UV | | Normal | | | | | |
| | 50° | C. | Normal | Normal | Normal | Normal | Normal | Normal |
| | 40° | C. | Normal | Normal | Normal | Normal | Normal | Normal |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 8 | UV | | Normal | | | | | |
| | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Example 9 | UV | | Normal | | | | | |
| | 50° | C. | Normal | Normal | Normal | Normal | Slight | Slight |
| | | | | | | | yellowing | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Comparative | UV | | Obvious yellowing | | | | | |
| Example 1 | 50° | C. | Normal | Normal | Normal | Yellowing | Yellowing | Yellowing |
| | | | | | | | deepened, | deepened, |
| | | | | | | | delamination | delamination |
| | 40° | C. | Normal | Normal | Normal | Yellowing | Yellowing | Yellowing |
| | | | | | | | deepened, | deepened, |
| | | | | | | | delamination | delamination |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Delamination | Delamination | Delamination |
| Comparative | UV | | Slight yellowing | | | | | |
| Example 2 | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Slight |
| | | | | | | | | delamination |
| Comparative | UV | | Obvious yellowing | | | | | |
| Example 3 | 50° | C. | Normal | Normal | Slight | Yellow | Yellow | Severe |
| | | | | | yellowing | deepened | deepened | yellowing |
| | 40° | C. | Normal | Normal | Normal | Slight | Yellow | Yellow |
| | | | | | | yellowing | deepened | deepened |
| | 25° | C. | Normal | Normal | Normal | Normal | Normal | Slight |
| | | | | | | | | yellowing |
| | 5° | C. | Normal | Normal | Normal | Normal | Normal | Normal |
| | −8° | C. | Normal | Normal | Normal | Water | Water | Water |
| | | | | | | exudation | exudation | exudation |
| Comparative | UV | | Delamination | | | | | |
| Example 4 | 50° | C. | Delamination | Delamination | Delamination | Delamination, | Delamination, | Delamination, |
| | | | | | | yellowing | yellow | severe |
| | | | | | | | deepened | yellowing |
| | 40° | C. | Delamination | Delamination | Delamination | Delamination, | Delamination, | Delamination, |
| | | | | | | yellowing | yellow | severe |
| | | | | | | | deepened | yellowing |
| | 25° | C. | Delamination | Delamination | Delamination | Delamination | Delamination | Delamination |
| | 5° | C. | Delamination | Delamination | Delamination | Delamination | Delamination | Delamination |
| | −8° | C. | Delamination | Delamination | Delamination | Delamination | Delamination | Delamination |
| Comparative | UV | | Normal | | | | | |
| Example 5 | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Comparative | UV | | Delamination | | | | | |
| Example 6 | 50° | C. | Delamination | Delamination | Delamination | Delamination, | Delamination, | Delamination, |
| | | | | | | yellowing | yellow | severe |
| | | | | | | | deepened | yellowing |
| | 40° | C. | Delamination | Delamination | Delamination | Delamination, | Delamination, | Delamination, |
| | | | | | | yellowing | yellow | severe |
| | | | | | | | deepened | yellowing |
| | 25° | C. | Delamination | Delamination | Delamination | Delamination | Delamination | Delamination |
| | 5° | C. | Delamination | Delamination | Delamination | Delamination | Delamination | Delamination |
| | −8° | C. | Delamination | Delamination | Delamination | Delamination, | Delamination, | Delamination, |
| | | | | | | water | water | water |
| | | | | | | exudation | exudation | exudation |
| Comparative | UV | | Normal | | | | | |
| Example 7 | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Comparative | UV | | Normal | | | | | |
| Example 8 | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |
| Comparative | UV | | Normal | | | | | |
| Example 9 | 50° | C. | Normal | Normal | Normal | Normal | Normal | Slight |
| | | | | | | | | yellowing |
| | 40° | C. | Normal | Normal | Normal | Normal | Normal | Slight |
| | | | | | | | | yellowing |
| | 25° | C. | Normal | Normal | Normal | Normal | Normal | Normal |
| | 5° | C. | Normal | Normal | Normal | Normal | Normal | Normal |
| | −8° | C. | Normal | Normal | Normal | Normal | Normal | Normal |

According to the data in Table 3, colors in Examples 1 and 6 were changed in UV and high temperature environment, but there was not much change. The changes of Example 1 at 50° C. and 40° C. were smaller than those of Example 6. Comparative example 1: under the conditions of UV, 50° C., 40° C., and −8° C., serious discoloration or unstable phenomena may occur, which indicated that the capsule shell components need to be within a certain ratio range to achieve a better wrapping effect and stability, and too much or too little will affect the stability. Comparative Example 2: in UV and high temperature environment, the color will change, but there was not much change, and there was slight delamination at low temperature, which indicated that the amount of active ingredients needs to be within a proper range to make the emulsion have better stability, and too much or too little will affect the stability. Comparative Example 3: under the conditions of UV, 50° C., and 40° C., there was serious discoloration, and after the temperature was restored at 15° C., there was water precipitated, and the stability was greatly changed. It showed that the inclusion obtained by using nonionic vesicles to wrap the active ingredients was more stable than the inclusion obtained by liposome encapsulation. In Comparative Example 4, simple mixing cannot form a good vesicle inclusion structure, and the stability was extremely poor. In Comparative example 6, delamination occurred under the conditions of low temperature, high temperature, and UV, which indicated that the capsule shell components had good wrapping effect and stability only within a certain ratio.

### Effect Example 2: Permeability and Irritation Soothing Experiments

A capsaicin pathway in human skin can be activated by capsaicin, acidity (pH<6.05), and temperature higher than 42° C. Zebrafish also has a capsaicin pathway, which can be activated at a high temperature, reaching a peak at 37° C. or above temperature, indicating that a swimming speed is significantly increased. Therefore, zebrafish activated by high temperature is very suitable for sting soothing tests. A movement distance of zebrafish embryos was measured by software, changes of the movement distance of the zebrafish embryos in a tested treatment group and a model control group were compared, and a reduction rate of the movement distance was calculated to evaluate the ability of raw materials or products to soothe irritation.

The inclusions in Examples 1 to 9 and in Comparative Examples 1 to 9 were tested under the following conditions.

Sample treatment: The inclusions in Examples 1 to 9 and in Comparative Examples 1 to 9 were homogenized and evenly mixed with water at a high speed according to a mass ratio of 0.25%, ultrasonic treatment was performed for 10 min, then fish embryo culture solution was added to prepare a required concentration, the solution was centrifuged at 6500 r/min for 10 min after shaking for 30 s, and the supernatant was taken for testing. The concentration of the test solution should ensure that a death rate of the zebrafish embryos was less than 10% (embryos without heartbeat characteristics were defined as death), and three concentration groups were set during testing.

Test steps: healthy zebrafish embryos 5 days after fertilization were selected. The test needs to set up a blank control group (a fish embryo culture solution) and a sample group (a test sample). 16 fish embryos were randomly selected and transferred to a 96-well plate, with each well containing one fish embryo and 0.2 mL of test solution, and the blank control group was pure zebrafish culture solution. Place them in a thermostat at 28±1° C. for 2 hours. Then transfer them to a zebrafish motion recorder, and record a swimming track of each zebrafish embryo within 5 minutes in a dark environment by an infrared tracking software. Replace 0.18 mL of the solution in each well with fish embryo culture solution, and then put them in a thermostat at 28±1° C. for 24±1 h to count a survival rate of the fish embryos.

Data processing: read a pixel intensity of each well as the movement distance of each fish embryo.

Calculate a reduction rate of the movement distance: reduction rate=(C−T)/C×10000.

In the formula: T represents an average value of the movement distance of fish embryos in the test treatment group; C represents an average value of the movement distance of fish embryos in the blank control group; and the results were shown in Table 4.

TABLE 4
Test results of movement distance reduction rate

| Test object | Movement distance reduction rate (%) |
|---|---|
| Example 1 | 99.73% |
| Example 2 | 83.72% |
| Example 3 | 85.34% |
| Example 4 | 73.56% |
| Example 5 | 78.02% |
| Example 6 | 92.87% |
| Example 7 | 89.42% |
| Example 8 | 93.06% |
| Example 9 | 92.39% |
| Comparative Example 1 | 63.42% |
| Comparative Example 2 | 21.47% |
| Comparative Example 3 | 42.70% |
| Comparative Example 4 | 32.47% |
| Comparative Example 5 | 42.36% |
| Comparative Example 6 | 52.96% |
| Comparative Example 7 | 23.27% |
| Comparative Example 8 | 67.32% |
| Comparative Example 9 | 47.63% |
| Blank group | 100% |

The test results were shown in Table 4, the movement distance reduction rate of the blank group was 100%, the movement distance reduction rate of the zebrafish in Example 1 was 99.730%, and the movement distance reduction rate of the inclusions in Examples 1 to 9 was higher than that of the inclusions in Comparative Examples 1 to 9, indicated that the high-permeability inclusion for soothing skin redness, heat, and pain of this disclosure has a powerful soothing effect on TRPV1 and can reduce skin irritation.

The movement distance reduction rate in Comparative Example 1 and Comparative Example 6 was decreased, indicating that the components of capsule shell need to be within a reasonable range to make the inclusion have a good permeability and soothing effect. The movement distance reduction rate in Comparative Example 2 and Comparative Example 7 was significantly decreased, indicating that the active ingredients need to be in a proper range to make the inclusion have a good permeability and soothing effect. The movement distance reduction rate in Comparative Example 4 was 32.47%, indicating that the soothing effect of the 4-tert-butylcyclohexanol, crocus sativus extract, Tephrosia purpurea (L.) Pers. seed extract, and Kadsura longipedunculata Finet & Gagnep. extract without wrapping on zebrafish was weaker than that of the composition wrapped with nonionic vesicles. After wrapping, the permeability of each active ingredient increases, and more active ingredients can permeate the skin to enhance the soothing effect. The movement distance reduction rate in Comparative Example 5 was 42.36%, less than that in Example 1, indicating that the 4-tert-butylcyclohexanol and the crocus sativus extract can be encapsulated in the capsule shell layer together to exert their instant soothing effect and better inhibit the skin redness, heat, and pain caused by the activation of TRPV1. In Comparative Examples 8 and 9, although the amount of active ingredients was the same, four components were not compounded, and the synergistic effect between components was weakened, resulting in a poor soothing effect of the inclusion.

### Effect Example 3: Instant Soothing and Redness Fading Efficacy Experiment

The emulsions obtained in Examples 1 to 9 and Comparative Examples 1 to 9 were tested for instant soothing and redness fading efficacy under the following conditions, and test results were shown in Table 5.

On the day of the visit, 72 volunteers were not allowed to apply any products to test areas. After washing their arms, they sat still for 20 minutes in an air-conditioned room at a temperature of 21±1° C. and a humidity of 50±10% and were randomly divided into 9 groups with 8 people in each group. Two areas of 10 cm² (2.5 cm×4 cm) were selected on left and right forearms of the volunteers, and marked with a skin marker, and initial TEWL values and a* values of the selected arm areas were measured by a transepidermal water loss probe of skin, that is the TewaMeter® TM Hex (Courage+Khazaka), and a Colorimeter CL400.

The test model used a 3M medical adhesive tape tearing model: that is, a 3M medical PE adhesive tape is used to carry out tear and damage treatment on the measured area, and a skin damaged model is established. After the damaged model is established 30 minutes later, the TEWL value and the a* value were measured, and a difference between the TEWL value and the a* value in different injured areas of the hand was kept at ±3 g/hm². The test emulsions obtained in Examples 1 to 9 and Comparative Examples 1 to 9 were distributed to 9 groups of the volunteers, and each person randomly selected 3 areas, respectively using two kinds of test emulsions and each area using a blank emulsion. After using products for 1 hour and 4 hours, the TEWL value and the a* value of the test areas were measured. The instant skin barrier repair ability of the two samples was represented by the change of the TEWL value, and the instant redness fading effect was represented by the change of the a* value. The formula was as follows:

$$Improvement rate ( \% ) = \frac{{X}_{Repair node} - {X}_{Model establishment node}}{{X}_{Repair node} - {X}_{Initial node}} \times 1 \; 00 \% \times - 1.$$

TABLE 5
Instant Soothing and Redness fading Effects
of Examples and Comparative Examples

| | |
|---|---|
| TEWL improvement | a* value improvement |
| rate (%) | rate (%) |

| Test object | 1 h | 4 h | 1 h | 4 h |
|---|---|---|---|---|
| Example 1 | 37.32% | 42.67% | 19.80% | 23.72% |
| Example 2 | 32.54% | 35.47% | 13.65% | 17.42% |
| Example 3 | 31.93% | 37.42% | 15.32% | 19.03% |
| Example 4 | 23.65% | 27.32% | 12.04% | 15.37% |
| Example 5 | 25.32% | 31.89% | 13.24% | 16.39% |
| Example 6 | 33.21% | 37.21% | 15.32% | 21.39% |
| Example 7 | 23.51% | 33.97% | 16.23% | 19.87% |
| Example 8 | 35.92% | 40.75% | 17.28% | 22.89% |
| Example 9 | 34.83% | 40.32% | 18.32% | 22.04% |
| Comparative | 17.42% | 25.32% | 6.03% | 12.83% |
| Example 1 | | | | |
| Comparative | 17.38% | 28.04% | 12.04% | 17.32% |
| Example 2 | | | | |
| Comparative | 19.03% | 26.39% | 7.32% | 15.01% |
| Example 3 | | | | |
| Comparative | 21.67% | 27.31% | 14.97% | 17.32% |
| Example 4 | | | | |
| Comparative | 20.95% | 25.32% | 15.78% | 19.23% |
| Example 5 | | | | |
| Comparative | 18.42% | 25.04% | 5.03% | 9.32% |
| Example 6 | | | | |
| Comparative | 15.68% | 27.06% | 14.83% | 15.04% |
| Example 7 | | | | |
| Comparative | 16.94% | 31.05% | 11.95% | 20.95% |
| Example 8 | | | | |
| Comparative | 15.20% | 30.27% | 12.31% | 17.03% |
| Example 9 | | | | |

The data in Table 5 demonstrate that, compared with Comparative Examples 1 to 9, Examples 1 to 9 have remarkable effects on instantly improving the barrier damage and redness of skin, among which the improvement rates within 4 h of the TEWL and a* values of Example 1 are better than those of Examples 2 to 6. In Comparative Examples 1 and 6, the composition ratio of the capsule shell is not good, which leads to the decrease of wrapping and stability of the inclusion. In Comparative Examples 2 and 7, the ratio of active ingredients is not good, which leads to the decrease of the instant soothing and redness fading effects of the inclusion. In Comparative Example 3, lecithin is used to encapsulate the active ingredients, which leads to poor stability of the inclusion. In Comparative Example 4, although the active ingredients were not wrapped, it had a certain redness fading effect, but the instant soothing effect within 1 hour was not good. In Comparative Example 5, the crocus sativus extract was not wrapped in the capsule shell, which resulted in that the crocus sativus extract could not be released preferentially, resulting in poor instant soothing effect. In Comparative Example 8, although the total amount of active ingredients was the same, but the crocus sativus extract was not added, the soothing effect was not good. In Comparative Example 9, although the total amount of active ingredients was the same, but the 4-tert-butylcyclohexanol was not added, the soothing effect was not good.

The data in Table 5 showed that the high-permeability inclusion of this disclosure had excellent instant soothing and redness fading effects.

### Effect Example 4: Anti-Inflammatory and Soothing Experiment

In vitro stimuli can activate toll-like receptors (TLR-2 and TLR-4) on keratinocytes, leading to the activation of MAPK and NF-κB pathways. Then, the keratinocytes produce IL-1, IL-6, IL-8, TNF-α, and human HBD-2 (defensins), which induces inflammation and causes skin discomfort. The anti-inflammatory effects of the inclusions in Examples 1 to 9 and Comparative Examples 1 to 9 were evaluated by using LPS-induced HaCaT cells as an inflammatory cell model in vitro and detecting inflammatory factors IL-la, IL-10, and TNF-α secreted in cell supernatant by an ELISA kit.

The HaCaT cells were inoculated into a 12-well plate with a density of 2.0-3.0×10⁵ cells/well, and incubated in an incubator at a temperature of 37±1° C. and a humidity of 90±5%. After the incubation, the culture plate was taken out, and the original culture solution in the well was discarded. In the sample group, 1 mL of test samples with a volume ratio of 0.3% and 50 g/ml of LPS culture solution were added to each well; in the model group, only 50 g/mL of LPS culture solution were added; in the positive control group, 1 mL of dexamethasone (50 g/mL) and LPS (50 g/mL) culture solution was added, while in the blank negative control group, 1 mL of culture solution was only added for culturing 24 h. The supernatant was collected by centrifugation and stored at −80° C. Cytokines of IL-la, IL-8, and TNF-α were determined by using the method described in the ELISA kit, and signals were detected at a wavelength of 450 nm. The content of inflammatory factors was shown in Table 6 by the secretion amount (pg) of IL-i1α, IL-1β, and TNF-α contained in each mL of the culture solution, and triplicate wells were set for each test.

TABLE 6
Comparison of differences in inflammatory factors in
different samples (Mean ± SD, n = 3)

| Group | IL-1α (pg/Ml) | IL-1β (pg/mL) | TNF-α (pg/mL) |
|---|---|---|---|
| Negative control | 42.28 ± 0.38 | 34.71 ± 0.42* | 79.36 ± 4.48* |
| group (NT) | | | |
| Model control | 63.29 ± 0.39 | 66.29 ± 0.29* | 92.33 ± 2.25* |
| group (M) | | | |
| Dexamethasone | 36.30 ± 0.21 | 24.67 ± 0.65* | 77.27 ± 2.96* |
| group (PC) | | | |
| Example 1 | 24.31 ± 0.92* | 26.39 ± 0.52* | 32.57 ± 0.91* |
| Example 2 | 27.68 ± 0.27* | 28.03 ± 0.84* | 43.4 ± 0.91* |
| Example 3 | 32.41 ± 0.15* | 27.03 ± 0.34* | 42.73 ± 0.15* |
| Example 4 | 35.2 ± 0.71* | 36.32 ± 0.73* | 53.28 ± 0.11* |
| Example 5 | 33.29 ± 0.49* | 33.75 ± 0.72* | 55.37 ± 0.86* |
| Example 6 | 37.42 ± 0.84* | 32.39 ± 0.88* | 51.76 ± 0.52* |
| Example 7 | 28.32 ± 0.85* | 27.3 ± 0.82* | 37.39 ± 0.77* |
| Example 8 | 29.31 ± 0.69* | 28.04 ± 0.05* | 36.41 ± 0.88* |
| Example 9 | 27.03 ± 0.47* | 29.94 ± 0.14* | 35.48 ± 0.72* |
| Comparative | 54.26 ± 0.52* | 44.73 ± 0.45* | 78.42 ± 0.91* |
| Example 1 | | | |
| Comparative | 62.36 ± 0.31 | 64.27 ± 0.57 | 90.45 ± 0.96* |
| Example 2 | | | |
| Comparative | 53.26 ± 0.65* | 47.36 ± 0.29* | 63.2 ± 0.84* |
| Example 3 | | | |
| Comparative | 46.02 ± 0.4* | 41.26 ± 0.2* | 63.78 ± 0.58* |
| Example 4 | | | |
| Comparative | 48.35 ± 0.75* | 39.25 ± 0.18* | 60.36 ± 0.85* |
| Example 5 | | | |
| Comparative | 44.27 ± 0.28* | 42.38 ± 0.65* | 57.11 ± 0.81* |
| Example 6 | | | |
| Comparative | 63.25 ± 0.87 | 68.42 ± 0.84 | 95.35 ± 0.18 |
| Example 7 | | | |
| Comparative | 57.36 ± 0.24* | 53.27 ± 0.96* | 72.36 ± 0.55* |
| Example 8 | | | |
| Comparative | 60.27 ± 0.1* | 54.32 ± 0.57* | 81.28 ± 0.17* |
| Example 9 | | | |

Note:
*indicates that the secretion amount of inflammatory factors in the sample is significantly different from that in the model group, P < 0.05.

The data in Table 6 demonstrate that, compared with Comparative Examples 1 to 9, Examples 1 to 9 have stronger ability to inhibit the secretion of inflammatory factors, among which Example 1 has the best inhibitory effect on the secretion of inflammatory factors. It can be seen from analysis of Comparative Examples 1, 6, and 3 that both the poor ratio of the capsule shell components and the use of lecithin wrapping can hinder the ability of inhibiting the secretion of inflammatory factors; it can be seen from analysis of Comparative Examples 2 and 7 that the poor ratio of the active ingredients can lead to the weakening of the ability of soothing inflammation of the inclusion; however, the active ingredients were not wrapped in Comparative Example 4, although it also has a certain inhibitory ability on inflammatory factors, but there was still a gap in the inhibitory ability compared with Examples; the crocus sativus extract was not wrapped in the capsule shell in Comparative example 5, which also has some influence on the inhibitory ability of inflammatory factors; and the absence of the crocus sativus extract in Comparative Example 8 and the absence of 4-tert-butylcyclohexanol in Comparative Example 9 both resulted in poor inhibitory effect on inflammatory factors.

### Effect Example 5: Long-lasting soothing experiment

The emulsions obtained in Example 1, Comparative Example 4, and Comparative Examples 8 to 9 were tested for long-lasting soothing under the following conditions. The improvement effects of TEWL, redness, and lactic acid sting were shown in Table 7.

Selection of volunteers: 15 people in each group, aged between 21 and 44, who have no serious skin history, no cosmetic allergy history, are sensitive (capsaicin stimulation is positive), with TEWL between 20 g/m² h and 30 g/m² h were selected, and there are 4 groups in total.

Test steps: before using the sample, all volunteers uniformly used amino acid facial cleanser to clean their faces, sat quietly in an efficacy evaluation room at constant temperature and humidity for more than 5 minutes, and measured the facial TEWL value and a* value as DO data with a transepidermal moisture tester and a skin color tester; 50 μL of 10% lactic acid aqueous solution was smeared on the facial nasolabial sulcus of subjects, and the subjects were asked about their sensation symptoms at 2.5 min and 5 min respectively, to score according to a 4-point scale (0 represents no sting, 1 represents mild sting, 2 represents moderate sting, and 3 represents severe sting); the sum of the two scores was taken as a score result of the lactic acid sting test as the DO data, and the emulsions of Example 1, Comparative Example 4, and Comparative Examples 8 to 9 were used once in the morning and evening, with a same use amount; and after 14 days and 28 days, the facial TEWL, a* value, and lactic acid stimulation values were measured respectively, and the influence of the samples on skin physiological parameters was indicated by numerical changes.

TABLE 7
Results of 28-day long-lasting soothing efficacy test

| | TEWL value | | | a* value | | | Lactic acid sting | | |
|---|---|---|---|---|---|---|---|---|---|
| | | 2 | 4 | | 2 | 4 | | 2 | 4 |
| | Before | weeks | weeks | Before | weeks | weeks | Before | weeks | weeks |
| Test object | use | later | later | use | later | later | use | later | later |
| Example 1 | 25.13 | 20.45 | 17.71 | 14.08 | 12.48 | 9.06 | 4.2 | 2.1 | 0.6 |
| Comparative | 26.96 | 21.97 | 19.28 | 13.87 | 12.28 | 10.18 | 3.9 | 2.7 | 1.9 |
| Example 4 | | | | | | | | | |
| Comparative | 26.32 | 23.41 | 22.89 | 14.32 | 13.09 | 11.27 | 4.3 | 3.7 | 2.3 |
| Example 8 | | | | | | | | | |
| Comparative | 25.41 | 23.78 | 21.93 | 14.03 | 12.93 | 10.97 | 4.3 | 3.5 | 2.5 |
| Example 9 | | | | | | | | | |

Compared with the comparative examples, the emulsion of the examples can obviously improve the skin condition and effectively soothe the redness, heat, and pain of the skin in the 28-day TEWL, a* value, and lactic acid sting test.

Compared with other examples, Example 1 has the best long-lasting soothing effect. The data in Table 7 demonstrate that, in Comparative Example 4, the active ingredients were not wrapped, so that the permeability of the active ingredients decreased, and the long-lasting soothing effect was poor. In Comparative Example 8, although the total amount of active ingredients was the same, but the crocus sativus extract was not added, so the synergistic effect between the active ingredients was reduced and the long-lasting soothing effect was not good. In Comparative Example 9, although the total amount of active ingredients was the same, but the 4-tert-butylcyclohexanol was not added, so the synergistic effect between the active ingredients was reduced and the long-lasting soothing effect was not good.

The data in Table 7 showed that the inclusion composition wrapped with nonionic vesicles can effectively soothe the redness, heat, and pain of the skin, and has a certain barrier repairing function.

Finally, it should be noted that the foregoing embodiments are merely intended to describe the technical solutions of this disclosure, but not intended to constitute any limitation. Although this disclosure is described in detail with reference to preferred embodiments, it will be understood by those of ordinary skill in the art that modifications or equivalent substitutions may be made to the technical solutions of this disclosure without departing from the essence and scope of the technical solutions of this disclosure.

What is claimed is:

1. An inclusion for soothing skin redness, heat, and pain, wherein the inclusion comprises a capsule shell and capsule contents that are encapsulated in a hollow cavity of the capsule shell;
the capsule shell comprises a nonionic surfactant, a cosurfactant, cholesterol, 4-tert-butylcyclohexanol, a crocus sativus extract, and water;
the capsule contents comprise a Tephrosia purpurea (L.) Pers. seed extract and a Kadsura longipedunculata Finet & Gagnep. extract;
in parts by weight, content of each ingredient in the inclusion is as follows:
a mass sum of the nonionic surfactant, the cosurfactant, and the cholesterol is 1.6-16.5 parts;
a mass sum of the 4-tert-butylcyclohexanol, the crocus sativus extract, the Tephrosia purpurea (L.) Pers. seed extract, and the Kadsura longipedunculata Finet & Gagnep. extract is 0.4-10 parts; and the water is 0.2-4 parts;
a mass ratio of the nonionic surfactant, the cosurfactant, and the cholesterol is (1-12):(0.1-1.5):(0.5-3); and
a mass ratio of the 4-tert-butylcyclohexanol, the crocus sativus extract, the Tephrosia purpurea (L.) Pers. seed extract, and the Kadsura longipedunculata Finet & Gagnep. extract is (0.1-3):(0.1-2):(0.1-2):(0.1-3);
the nonionic surfactant comprises at least one of monoglyceride, propylene glycol fatty acid esters, polyglyceryl-10 oleate, polyglyceryl-10 diisostearate, polyglyceryl-3 methylglucose distearate, polyglyceryl-10 laurate, glyceryl oleate citrate, polyglyceryl-2 dipolyhydroxystearate, cetearyl alcohol, and cetearyl glucoside; and
the cosurfactant comprises at least one of polyethylene glycol-400, polyethylene glycol-600, caprylic/capric triglyceride, PEG-40 hydrogenated castor oil, and PEG-60 hydrogenated castor oil.

2. The inclusion for soothing skin redness, heat, and pain according to claim 1, wherein the mass ratio of the 4-tert-butylcyclohexanol, the crocus sativus extract, the Tephrosia purpurea (L.) Pers. seed extract, and the Kadsura longipedunculata Finet & Gagnep. extract is (0.5-1):(0.5-1):(0.1-1):(0.5-1).

3. The inclusion for soothing skin redness, heat, and pain according to claim 2, wherein the mass ratio of the 4-tert-butylcyclohexanol, the crocus sativus extract, the Tephrosia purpurea (L.) Pers. seed extract, and the Kadsura longipedunculata Finet & Gagnep. extract is 1:0.5:0.75:0.5.

4. The inclusion for soothing skin redness, heat, and pain according to claim 1, wherein the mass ratio of the nonionic surfactant, the cosurfactant, and the cholesterol is 6:0.75:1.5.

5. The inclusion for soothing skin redness, heat, and pain according to claim 1, wherein at least one of the following is met:
(2-1) the nonionic surfactant is the monoglyceride, the cetearyl alcohol, or the polyglyceryl-10 oleate; and
(2-2) the cosurfactant is the PEG-40 hydrogenated castor oil.

6. The inclusion for soothing skin redness, heat, and pain according to claim 5, wherein a mass ratio of the monoglyceride, the polyglyceryl-10 oleate, and the cetearyl alcohol is 3:2:1.

7. A preparation method of the inclusion for soothing skin redness, heat, and pain according to claim 1, comprising the following steps:
S1: stirring the 4-tert-butylcyclohexanol, the crocus sativus extract, the nonionic surfactant, and the cosurfactant under a heating condition until they were completely mixed, and cooling them to obtain a nonionic surfactant mixture;
S2: mixing the nonionic surfactant mixture obtained in step S1 with water and cholesterol, and performing heating, homogenizing, and cooling on them to obtain capsule materials; and
S3: adding the Tephrosia purpurea (L.) Pers. seed extract and the Kadsura longipedunculata Finet & Gagnep. extract into the capsule materials obtained in step S2, and homogenizing them under high pressure to obtain the inclusion for soothing skin redness, heat, and pain.

8. The preparation method of the inclusion for soothing skin redness, heat, and pain according to claim 7, wherein at least one of the following is met:
(3-1) in step S1, a heating temperature is 60° C. to 70° C.;
(3-2) in step S2, a heating temperature is 70° C. to 80° C., and a rotating speed during homogenization is 1500 r/min to 2500 r/min; and
(3-3) in step S3, a pressure of the high-pressure homogenization is 500 bar to 800 bar, a rotating speed is 1000 r/min to 2000 r/min, and a number of rotating times is 3 to 5.

9. Use of the inclusion for soothing skin redness, heat, and pain according to claim 1 in cosmetics.

10. An emulsion, wherein the emulsion comprises the inclusion for soothing skin redness, heat, and pain according to claim 1, and a mass percentage of the inclusion for soothing skin redness, heat, and pain in the emulsion is 3.2%-27%.
